(19) United States
(12) Patent Application Publication
(10) Pub. No.: US 2023/0233201 A1
(43) Pub. Date: Jul. 27, 2023

(54) APPARATUS AND METHOD FOR ENDOSCOPICALLY CLOSING GASTROINTESTINAL DEFECTS

(71) Applicant: Covidien LP, Mansfield, MA (US)

(72) Inventors: Hillary K. Huszar, Redwood City, CA (US); Ellie K. Ahlquist, San Jose, CA (US); Ryan W. Bertwell, San Jose, CA (US); Shawn C. Daniel, Mountlake Terrace, WA (US); William J. Camisa, Santa Clara, CA (US)

(21) Appl. No.: 18/100,182

(22) Filed: Jan. 23, 2023

Related U.S. Application Data
(60) Provisional application No. 63/302,210, filed on Jan. 24, 2022.

(51) Int. Cl. A61B 17/04

(52) U.S. Cl. CPC A61B 17/0401; A61B 17/0469; A61B 2017/0409

(57) ABSTRACT

A surgical system for use in repairing a wound after an endoscopic resection procedure includes a plurality of anchors coupled to one another via a tether. Each of the anchors includes a head defining an opening for receipt of the tether, and two or more tines configured to penetrate tissue. The tines are resiliently biased toward a deployed configuration, in which the tines project outwardly relative to the head and assume an arcuate shape.

### CROSS-REFERENCE TO RELATED APPLICATIONS

This application claims the benefit of the filing date of provisional U.S. Patent Application No. 63/302,210 filed on Jan. 24, 2022.

### FIELD

The present technology is related generally to an apparatus for endoscopically closing a wound.

### BACKGROUND

Endoscopic resection, such as endoscopic submucosal dissection (ESD), endoscopic mucosal resection (EMR), peroral endoscopic myotomy (POEM), and full thickness resection, has been accepted as a first choice of the treatment for early stage GI carcinomas because of less invasiveness and lower cost. ESD, for example, allows for an en bloc resection and accurate histopathological diagnosis regardless of the size and location of a lesion or an existence of severe fibrosis at the submucosal layer.

After endoscopic resection, such as ESD, the submucosa or muscle layer may be exposed. For example, after an ESD, lesions in the mucosal and submucosal space are removed leaving an exposed area in the GI tract. Oftentimes, clinicians decide not to close the opening in the mucosal layer. However, there has been recent interest and evidence that closing the mucosal layer may lead to reduced complications.

### SUMMARY

In accordance with an aspect of the disclosure, a surgical system for use in repairing a wound after an endoscopic resection procedure is provided. The surgical system includes a plurality of anchors, and a tether fixed to at least one of the anchors. Each anchor includes a head defining an opening, and two or more tines configured to penetrate tissue. The tines are fabricated from a shape memory material such that the tines are resiliently biased toward a deployed configuration. In the deployed configuration, the tines project outwardly relative to the head and assume an arcuate shape. The tether passes through the opening of each anchor.

In aspects, the tines may be configured to transition from a stowed configuration, in which the tines are linear, to the deployed configuration.

In aspects, the tines may have a proximal-facing surface having a concave shape, and a distal-facing surface having a convex shape.

In aspects, each anchor may further include a main body. The head may project proximally from the main body, and the tines may project distally from the main body.

In aspects, the tines and the head may be monolithically formed with one another.

In aspects, the tines and the head may be fabricated from a single wire that extends through the main body.

In aspects, the tines may extend in opposite directions from one another and perpendicularly relative to a longitudinal axis defined by the main body.

In aspects, each of the tines may each have a traumatic distal tip configured for penetrating tissue.

In accordance with another aspect of the disclosure, a surgical system for use in repairing a wound after an endoscopic resection procedure is provided and includes a plurality of anchors each including a main body, a head projecting from a proximal end portion of the main body and defining an elongate slot, and two or more tines projecting from a distal end portion of the main body and configured to penetrate tissue. The tines are fabricated from a shape memory material such that the tines are resiliently biased from a stowed configuration toward a deployed configuration. In the stowed configuration, the tines are parallel with a longitudinal axis defined by the main body, and in the deployed configuration, the tines project radially outward from the main body.

In aspects, the surgical system may further include a suture fixed to at least one of the anchors and passing through the elongated slot of each anchor.

In aspects, the surgical system may further include an endoscopic tube. The anchors and the suture may be stowed within the endoscopic tube.

In accordance with a further aspect of the disclosure, a method of repairing a wound after an endoscopic resection procedure is provided. The method includes deploying a plurality of anchors into gastrointestinal tissue at an outer periphery of a wound. As such, the outer periphery of the wound is surrounded by the anchors, whereby at least two tines of each anchor penetrate the tissue while flaring radially outward relative to a body portion of the anchor. The method further includes retracting a suture that connects the anchors to one another, thereby closing the wound.

In aspects, deploying the anchors may include transitioning the tines of each anchor from a stowed configuration, in which the tines are linear, to an expanded configuration.

In aspects, the method may further include penetrating the gastrointestinal tissue with a distal tip of a hypotube. Deploying the anchors may include deploying the anchors from the hypotube while the hypotube is penetrating the gastrointestinal tissue.

### BRIEF DESCRIPTION OF THE DRAWINGS

The accompanying drawings, which are incorporated in and constitute a part of the specification, illustrate embodiments of the disclosure and, together with a general description of the disclosure given above as well as the detailed description of the embodiment or embodiments given below, serve to explain the principles of this disclosure.

FIG. 1 is a side, perspective view illustrating a surgical system including a plurality of anchors stored within a hypotube;

FIG. 2 is a perspective view illustrating an embodiment of an anchor of the surgical system of FIG. 1;

FIG. 3A is a front view illustrating another embodiment of an anchor of the surgical system of FIG. 1;

FIG. 3B is a front view illustrating the anchor of FIG. 3A deployed in tissue; and

FIG. 4 illustrates the plurality of anchors of FIG. 1 used in repairing a wound formed from an endoscopic submucosal dissection surgical procedure.

### DETAILED DESCRIPTION

As used herein, the term “distal” refers to the portion that is being described which is further from a clinician, while the term “proximal” refers to the portion that is being described which is closer to a clinician. As used herein, the terms parallel and perpendicular are understood to include relative configurations that are substantially parallel and substantially perpendicular up to about +/−10 degrees from true parallel and true perpendicular. Further, to the extent consistent, any of the aspects described herein may be used in conjunction with any or all of the other aspects described herein.

The present disclosure is generally directed to a surgical system used in performing an endoscopic submucosal dissection. The surgical system includes a plurality of anchors configured for insertion through a working channel of an endoscope. Each of the anchors has a head and at least two tines that are resiliently biased toward an expanded state. The anchors are attached to one another via a suture and are configured for placement around a tissue defect caused by a tissue resection procedure. When the tines of the anchors are embedded in tissue, the heads of each of the anchors are exposed with the suture threaded therethrough. The end or ends of the suture is pulled, thereby closing the defect. In aspects, the anchors may be magnetically attracted to one another. These and other aspects of the present disclosure are described in greater detail below.

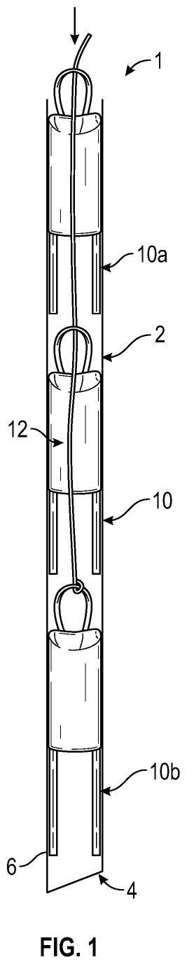

FIG. 1 illustrates a surgical system 1 including a hand-held surgical instrument 2 for deploying a plurality of anchors 10 (or tacks) into submucosal tissue. The surgical instrument 2 may be an endoscopic hypotube configured for passage through a working channel of an endoscope (not shown). The hypotube 2 defines a channel 4 therethrough having the anchors 10 slidably supported therein. The hypotube 2 may have a traumatic or sharp distal tip 6 configured for penetrating tissue. A pusher (not shown) may be positioned within the channel 4 of the hypotube 2 and behind the proximal-most anchor 10a. The pusher may be configured to be advanced by an actuation of a trigger (not shown) by a clinician to advance a single anchor 10 into tissue. A cutter, such as, a knife blade (not shown), may be positioned within the channel 4 of the hypotube 2 and configured to sever a tether 12, which is attached to each of the anchors 10, when the last anchor 10a is deployed. In some aspects, instead of the anchors 10 being deployable from a hand-held instrument, the anchors 10 may be deployed from a surgical robotic arm.

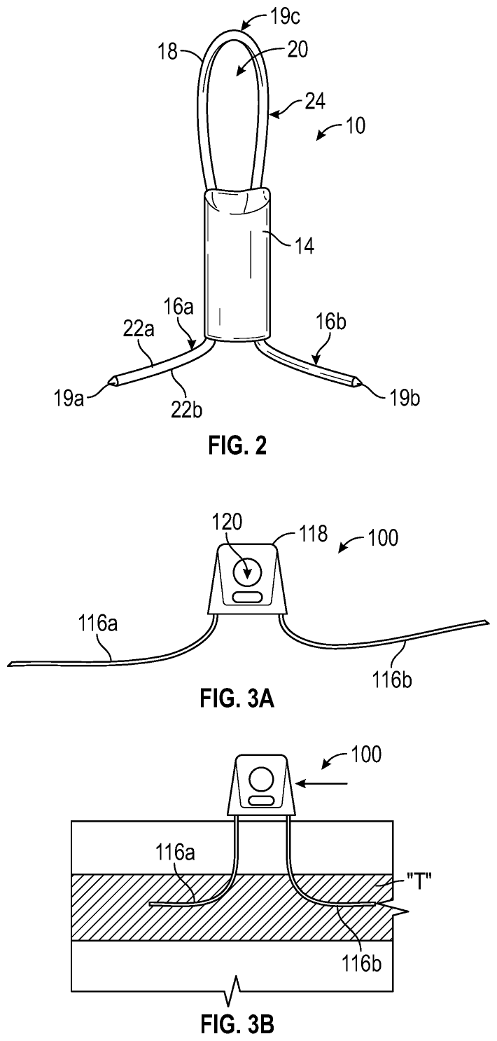

With reference to FIG. 2, each of the anchors 10 includes a main body 14 and at least two tines 16a, 16b (or spikes) extending distally from the main body 14. In aspects, the anchors 10 may each include more than two tines 16a, 16b, such as, for example, three or four tines that protrude radially outward from the main body 14 and being circumferentially spaced from one another. The main body 14 may be fabricated from metal (e.g., stainless steel) and may have a cylindrical or tubular shape. In aspects, other materials for fabricating the main body 14 and other shapes for the main body 14 are also contemplated. The anchor 10 may have a head 18 protruding proximally from the main body 14 and defining an opening or slot 20 configured for receipt of the tether 12. The opening 20 may have an elongated shape or a circular shape.

The tines 16a, 16b of each of the anchors 10 are fabricated from a shape memory material (e.g., nickel titanium) such that the tines 16a, 16b are resiliently biased toward an expanded or deployed configuration (FIG. 2). When the anchors 10 are stowed within the hypotube 2, the tines 16a, 16b, against the resilient bias thereof, are forced into a linear shape in which the tines 16a, 16b are parallel with the longitudinal axis of the main body 14. In the deployed configuration, the tines 16a, 16b protrude distally and flare outwardly from the main body 14. The tines 16a, 16b each have a generally proximal-facing surface 22a having a concave shape, and a generally distal-facing surface 22b having a convex shape, which together assist in securing the tines 16a, 16b in tissue. The tines 16a, 16b may extend perpendicularly (e.g., from about 70 to about 100 degrees) relative to a longitudinal axis of the main body 14. Each of the tines 16a, 16b have traumatic or sharp distal tips 19 configured to penetrate tissue.

It is contemplated that the head 18 of the anchor 10 and the tines 16a, 16b of the anchor 10 are monolithically formed with one another from a single metal wire 24, such as, for example, a wire fabricated from a shape memory material (e.g., nickel titanium). To manufacture the anchor 10, two opposing ends 19a, 19b of the wire 24 may be passed distally through the main body 14 with a middle portion 19c of the wire 24 extending proximally from the main body 14 and forming the head 18 of the anchor 10, and the ends 19a, 19b of the wire 24 forming the tips of the tines 16a, 16b which project distally from the main body 14. The wire 24 may be fixed to the main body 14 (e.g., crimped or welded) to fix the positions of the tines 16a, 16b and the head 19, or in some aspects the wire 24 may be slidable relative to the main body 14 to allow for the selective adjustment of the length of the tines 16a, 16b and height of the slot 20 of the head 18.

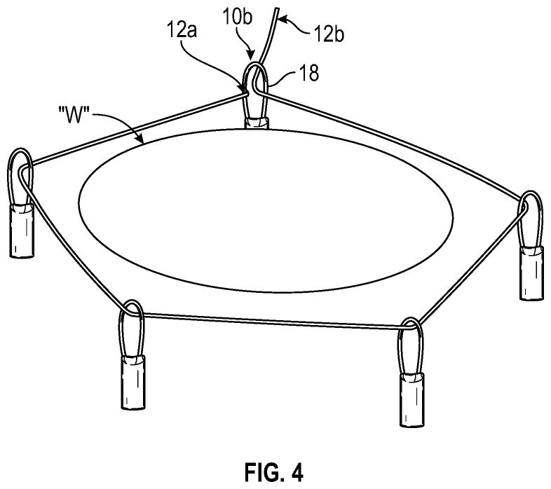

The tether 12 (FIG. 1) has a first end 12a (FIG. 4) fixed to an anchor, for example, a distal-most anchor 10b within the hypotube 2, and a free second end 12b. The tether 12 is threaded through each of the openings 20 of the anchors 10. The free second end 12b of the tether 12 may be manipulated by a surgical tool, such as, a tissue grasper, to pull on the tether 12. In aspects, the tether 12 may be a suture, a single strand tether, a flexible wire, thread, cable, a polymer or rubber strand, or the like. The tether 12 may be fabricated from propylene and/or coated with a lubricious material, such as silicone.

With reference to FIGS. 3A and 3B, another embodiment of a tissue anchor 100 is illustrated, similar to tissue anchors 10. The tissue anchor 100 may be used in place of or in addition to anchors 10. The tissue anchor includes a head 118 defining a suture opening 120 and at least two tines 116a, 116b extending directly from the head 118. The tines 116a, 116b are resiliently biased toward a deployed configuration (FIGS. 3A, 3B) in which the tines 116a, 116b flair radially outward and project distally from the head 118. When the tines 116a, 116b are deployed into tissue “T” (FIG. 3B), the tissue “T” may prevent the tines 116a, 116b from assuming their fully deployed configuration.

In use, during an endoscopic resection, such as ESD, lesions in the mucosal and submucosal space are removed leaving an exposed area in the GI tract. To close the exposed area of wound “W” (FIG. 4) in the GI tract, the surgical system 1 of the present disclosure may be utilized. More specifically, the hypotube 2, with the plurality of anchors 10 stowed therein, is passed through a working channel of an endoscope and the distal tip 6 of the hypotube 2 is positioned adjacent an outer periphery of the wound “W” and marginally within the tissue surface. The distal-most anchor 10b may be deployed from the hypotube 2, whereby the tines 16a, 16b of the anchor 10, upon exiting the distal tip 6 of the hypotube 2, transition from a stowed configuration, in which the tines 16a, 16b are parallel with a longitudinal axis of the main body 14, to a deployed or expanded configuration, in which the tines 16a, 16b flare outwardly. As the anchor 10b is deployed from the hypotube 2, the tines 16a, 16b embed in the tissue to fix the anchor 10b adjacent the wound “W” with the head 18 exposed.

With the distal-most anchor 10b secured to the tissue, the hypotube 2 is moved laterally relative to the distal-most anchor 10b to another location of the outer periphery of the wound “W” and adjacent the distal-most anchor 10b. Another anchor 10 is deployed from the hypotube 2 to secure the anchor 10 in the tissue. Additional anchors 10 may be deployed from the hypotube 2 until the wound “W” is surrounded with anchors 10. The free end 12b of the tether 12 may be pulled or retracted, whereby the heads 18 of the anchors 10 along with the tines 16a, 16b of the anchors 10 constrict about the wound “W” and approximate one another to close the wound “W.” With the anchors 10 in a constricted state relative to one another, the free end 12b of the tether 12 is tied off to maintain the anchors 10 in the constricted state with the wound “W” closed.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

What is claimed is:

1. A surgical system for use in repairing a wound after an endoscopic procedure, the surgical system comprising:
a plurality of anchors, each anchor of the plurality of anchors including:
a head defining an opening; and
at least two tines configured to penetrate tissue, the at least two tines being fabricated from a shape memory material such that the at least two tines are resiliently biased toward a deployed configuration, in which the at least two tines project outwardly relative to the head and assume an arcuate shape; and
a tether fixed to at least one anchor of the plurality of anchors and passing through the opening of each anchor of the plurality of anchors.

2. The surgical system according to claim 1, wherein the at least two tines are configured to transition from a stowed configuration, in which the at least two tines are linear, to the deployed configuration.

3. The surgical system according to claim 1, wherein the at least two tines have a proximal-facing surface having a concave shape, and a distal-facing surface having a convex shape.

4. The surgical system according to claim 1, wherein each anchor of the plurality of anchors further includes a main body, the head projecting proximally from the main body, and the at least two tines project distally from the main body.

5. The surgical system according to claim 4, wherein the at least two tines and the head are monolithically formed with one another.

6. The surgical system according to claim 5, wherein the at least two tines and the head are fabricated from a single wire that extends through the main body.

7. The surgical system according to claim 4, wherein the at least two tines extend in opposite directions from one another and perpendicularly relative to a longitudinal axis defined by the main body.

8. The surgical system according to claim 1, wherein the at least two tines each have a traumatic distal tip configured for penetrating tissue.

9. A surgical system for use in repairing a wound after an endoscopic resection procedure, the surgical system comprising:
a plurality of anchors, each anchor of the plurality of anchors including:
a main body;
a head projecting from a proximal end portion of the main body and defining an elongate slot; and
at least two tines projecting from a distal end portion of the main body and configured to penetrate tissue, the at least two tines being fabricated from a shape memory material such that the at least two tines are resiliently biased from a stowed configuration, in which the at least two tines are parallel with a longitudinal axis defined by the main body, toward a deployed configuration, in which the at least two tines project radially outward from the main body.

10. The surgical system according to claim 9, wherein the at least two tines have a proximal-facing surface having a concave shape, and a distal-facing surface having a convex shape.

11. The surgical system according to claim 9, wherein the at least two tines and the head are monolithically formed with one another.

12. The surgical system according to claim 11, wherein the at least two tines and the head are fabricated from a single wire that extends through the main body.

13. The surgical system according to claim 9, wherein the at least two tines extend in opposite directions from one another and perpendicularly relative to the longitudinal axis defined by the main body.

14. The surgical system according to claim 9, further comprising a suture fixed to at least one anchor of the plurality of anchors and passing through the elongated slot of each anchor of the plurality of anchors.

15. The surgical system according to claim 14, further comprising an endoscopic tube, the plurality of anchors and the suture being stowed within the endoscopic tube.

16. A method of repairing a wound after an endoscopic resection procedure, the method comprising:
deploying a plurality of anchors into gastrointestinal tissue at an outer periphery of a wound such that the outer periphery of the wound is surrounded by the plurality of anchors, whereby at least two tines of each anchor of the plurality of anchors penetrates the tissue while flaring radially outward relative to a body portion of the anchor; and
retracting a suture that connects the plurality of anchors to one another, thereby closing the wound.

17. The method according to claim 16, wherein deploying the plurality of anchors includes transitioning the at least two tines of each anchor from a stowed configuration, in which the at least two tines are linear, to an expanded configuration.

18. The method according to claim 17, further comprising penetrating the gastrointestinal tissue with a distal tip of a hypotube, wherein deploying the plurality of anchors includes deploying the plurality of anchors from the hypotube while the hypotube is penetrating the gastrointestinal tissue.
